(19) United States
(12) Patent Application Publication
(10) Pub. No.: US 2024/0182393 A1
(43) Pub. Date: Jun. 6, 2024

(54) SELECTIVE CATALYTIC ALKENE ISOMERIZATION FOR MAKING FRAGRANCE INGREDIENTS OR INTERMEDIATES

(71) Applicant: INTERNATIONAL FLAVORS & FRAGRANCES INC., NEW YORK, NY (US)

(72) Inventors: ANTONIO LEYVA-PÉREZ, VALENCIA (ES); MARTA MON-CONEJERO, PATERNA (ES); SERGIO SANZ-NAVARRO, VALENCIA (ES)

(21) Appl. No.: 18/282,838

(22) PCT Filed: Mar. 18, 2022
(86) PCT No.: PCT/US22/20979

§ 371 Date: Sep. 19, 2023

(30) Foreign Application Priority Data
Mar. 23, 2021 (EP) 21382234.9

(51) Int. Cl. C07C 41/32; C07B 35/08; C07C 29/56; C11B 9/00

(52) U.S. Cl. CPC C07C 41/32; C07B 35/08; C07C 29/56; C11B 9/0015; C11B 9/0061; C07B 2200/09

(57) ABSTRACT

This disclosure relates to a process for making fragrance ingredient or fragrance intermediate which involves isomerizing a starting material comprising a terminal alkene to form a product comprising an internal alkene in the presence of a ruthenium catalyst at a temperature of at least about 120° C.

### BACKGROUND

### Field of the Disclosure

The present disclosure relates to a selective alkene isomerization process to convert a terminal alkene to an internal alkene by using a ruthenium catalyst and more particularly to a process for making fragrance ingredient or fragrance intermediate.

### Description of Related Art

Alkene isomerization reactions have been identified as one of the key transformations that affords either final fragrance ingredients or valuable synthetic intermediates. However, each of these processes require different catalyst and conditions. Furthermore, isomerizations need to provide maximum conversion and be highly selective. Structural differences among starting material, desired final product and isomeric byproducts are in certain cases minimal, making final purification and/or isolation challenging due to the very similar physicochemical properties.

The isomerization of alkenes allows to move the double bond within a molecule to the desired position with full atom economy. However, the reported metal catalysts for this reaction are generally very expensive and give unacceptable mixture of alkene products (for representative examples see: Science 2019, 363, 391-396; ChemCatChem 2017, 9, 3849-3859; M. Mayer, A. Welther, A. J. von Wangelin, ChemCatChem 2011, 3, 1567-1571; R. Uma, C. Crévisy, R. Grée, Chem. Rev. 2003, 103, 27-51; Y. Sasson, A. Zoran, J. Blum, J. Mol. Cat. 1981, 11, 293-300). In general, homogeneous Lewis acid catalysts are commonly employed in the Fine Chemical Industry for double bond isomerization reactions. Alternative catalysts include solid acid catalysts containing Brönsted, Lewis or both type of acid centers (for selected examples see: J. Catal. 1962, 1, 2231; EP211985A1; EP442159B1; US20150141720A1) such as sulfated-zirconias, metal (usually Pt) supported zeolites heteropolyacids, molybdenum oxides, Al₂O₃-TiO₂ and alkali exchanged (X type) or alumina doped (K, Na, Cs . . .) zeolites (see, for instance, U.S. Pat. No. 4,992,613A; Catal. Surv. Japan, 2002, 5, 81; WO9313038). However, these types of acid solids, nowadays widely used for skeletal isomerization in the petrochemical industry at high temperatures of 250-450° C. (see for instance: Ind. Eng. Chem., 1953, 45, 551-564; Synthesis, 1969, 97-112; Synth. Commun., 1997, 27, 4335-4340), are usually unsuitable for fine chemical products.

### BRIEF SUMMARY OF THE DISCLOSURE

The present disclosure provides an isomerization process for making fragrance ingredient or fragrance intermediate. The process converts a terminal alkene to an internal alkene and comprises isomerizing a starting material comprising a terminal alkene to form a product comprising an internal alkene in the presence of a ruthenium catalyst at a temperature of at least about 120° C. in a reaction zone.

### BRIEF DESCRIPTION OF THE FIGURES

Embodiments are illustrated in the accompanying figures to improve understanding of concepts as presented herein.

FIG. 1 shows molecular structures of some ruthenium complexes.

### DETAILED DESCRIPTION

The foregoing general description and the following detailed description are exemplary and explanatory only and are not restrictive of the invention, as defined in the appended claims. Other features and benefits of any one or more of the embodiments will be apparent from the following detailed description, and from the claims.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. For example, when a range of “1 to 10” is recited, the recited range should be construed as including ranges “1 to 8”, “3 to 10”, “2 to 7”, “1.5 to 6”, “3.4 to 7.8”, “1 to 2 and 7-10”, “2 to 4 and 6 to 9”, “1 to 3.6 and 7.2 to 8.9”, “1-5 and 10”, “2 and 8 to 10”, “1.5-4 and 8”, and the like.

While compositions and methods are described herein in terms of “comprising” various components or steps, the compositions and methods also can “consist essentially of” or “consist of” the various components or steps, unless stated otherwise.

Some alkene molecules may exist as cis or trans stereoisomers. Unless explicitly indicated, an alkene (molecule, structure, formula, or chemical name) as used herein includes both cis and trans stereoisomers, as well as any combinations or mixtures of the cis and trans stereoisomers.

Before addressing details of embodiments described below, some terms are defined or clarified.

The term “terminal alkene”, as used herein, means a molecule comprising an organic moiety represented by Formula I set forth below:

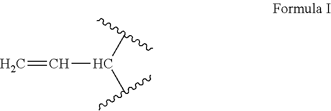 [SMILES: *C(*)C=C]

In a terminal alkene, the double bond is on a terminal carbon.

The term “internal alkene”, as used herein, means a molecule comprising a double bond which is not on the terminal carbon. In some embodiments, the internal alkene means a molecule comprising an organic moiety represented by Formula II set forth below:

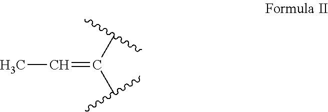 [SMILES: *C(*)=CC]

In some embodiments, the terminal alkene is a molecule comprising an organic moiety represented by Formula III set forth below wherein n is an integer from 1 to 20:

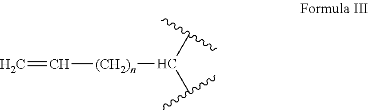

In such embodiments, the internal alkene is a molecule comprising an organic moiety represented by Formula IV, or a molecule comprising an organic moiety represented by Formula V, or mixtures of molecules of Formula IV and Formula V, as shown below:

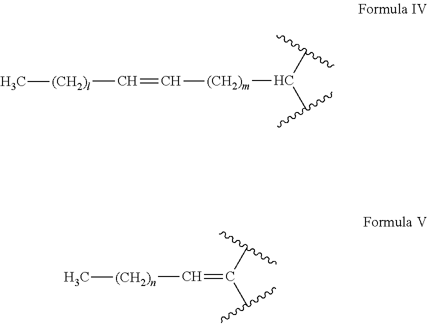

wherein l and m independently is an integer of 0 or greater than 0 (positive integer), and l+m=n−1. In some embodiments, n is an integer from 1 to 15, or an integer from 1 to 10. In some embodiments, n is 1.

Terminal alkene in this disclosure is a primary alkene, that is, the double bond is only connected to one carbon. The term “secondary alkene”, as used herein, means a molecule comprising a double bond which is connected to two carbons. The term “tertiary alkene”, as used herein, means a molecule comprising a double bond which is connected to three carbons. The term “quaternary alkene”, as used herein, means a molecule comprising a double bond which is connected to four carbons. A double bond in an aromatic functional group is not deemed as the kind of double bond referred to above in the definition of the terms secondary alkene, tertiary alkene and quaternary alkene.

The term “ruthenium complex”, as used herein, means a ruthenium coordination complex comprising a central ruthenium atom or cation surrounded by one or more coordination ligands that bind to the central ruthenium atom or cation. The bonding with the ruthenium atom or cation generally involves formal donation of one or more of the ligand's electron pairs.

The term “yield of the internal alkene”, as used herein, means the total molar amount of the internal alkene (product) formed in the isomerizing process (reaction) comparing with the total molar amount of the terminal alkene (starting material).

The term “fragrance intermediate”, as used herein, means an intermediate molecule (e.g., formed during multiple-step chemical reactions) which can react or be transformed to provide a final molecule which can be used as a fragrance ingredient.

In the process of this disclosure, a terminal alkene starting material is converted by an isomerization reaction to an internal alkene product. The terminal alkene starting material and the internal alkene product are position isomers, that is, they are different only on the position of the double bond. The isomerization process can be conducted by contacting a starting material comprising a terminal alkene with a ruthenium catalyst in a reaction zone. In some embodiments, the starting material comprises at least 80 wt %, or at least 85 wt %, or at least 90 wt %, or at least 95 wt %, or at least 98 wt %, or at least 99 wt % of the terminal alkene based on the total weight of the starting material. In some embodiments, the starting material consists essentially of or consists of the terminal alkene.

In some embodiments, the starting material comprises no more than 10 mol %, or no more than 5 mol %, or no more than 2 mol %, or no more than 1 mol %, or no more than 0.5 mol %, or no more than 0.2 mol %, or no more than 0.1 mol %, or no more than 0.05 mol %, or no more than 0.02 mol %, or no more than 0.01 mol % of a secondary alkene based on the total molar amount of the starting material. In some embodiments, the starting material is substantially free or free of a secondary alkene.

In some embodiments, the starting material comprises no more than 10 mol %, or no more than 5 mol %, or no more than 2 mol %, or no more than 1 mol %, or no more than 0.5 mol %, or no more than 0.2 mol %, or no more than 0.1 mol %, or no more than 0.05 mol %, or no more than 0.02 mol %, or no more than 0.01 mol % of a tertiary alkene based on the total molar amount of the starting material. In some embodiments, the starting material is substantially free or free of a tertiary alkene.

In some embodiments, the starting material comprises no more than 10 mol %, or no more than 5 mol %, or no more than 2 mol %, or no more than 1 mol %, or no more than 0.5 mol %, or no more than 0.2 mol %, or no more than 0.1 mol %, or no more than 0.05 mol %, or no more than 0.02 mol %, or no more than 0.01 mol % of a quaternary alkene based on the total molar amount of the starting material. In some embodiments, the starting material is substantially free or free of a quaternary alkene.

The terminal alkene molecule can comprise one or more functional groups containing oxygen and/or halide atoms. In some embodiments, the terminal alkene molecule comprises one or more functional groups selected from the group consisting of alkyl, aryl, alkoxy, hydroxy, halide and ester. In some embodiments, the terminal alkene molecule comprises one or more functional groups selected from the group consisting of alkyl, aryl, alkoxy and hydroxy.

In some embodiments, the terminal alkene molecule does not comprise nitrogen element. In some embodiments, the terminal alkene molecule does not comprise an organic basic group. In some embodiments, the terminal alkene molecule does not comprise an amine functional group.

In the process of this disclosure, a starting material comprising a terminal alkene is isomerized to form a product comprising an internal alkene in the presence of a ruthenium catalyst. The ruthenium catalyst is a ruthenium-containing catalyst which catalyzes the positional isomerization of the terminal alkene. Unless explicitly indicated, the ruthenium catalyst is not supported on a catalyst support or carrier. In some embodiments, the ruthenium catalyst is selected from the group consisting of ruthenium complexes, ruthenium salts, ruthenium in metal form, and mixtures thereof. In some embodiments, the ruthenium complexe is selected from the group consisting of ruthenium alkene complexes, ruthenium carbonyl complexes, ruthenium phosphine complexes, and mixtures thereof. In some embodiments, the ruthenium salt is selected from the group consisting of ruthenium chlorides, ruthenium bromides, ruthenium iodides, ruthenium oxides, ruthenium triflates, ruthenium perchlorates, and mixtures thereof. Ruthenium salts can be in anhydrous or hydrated form. Examples of ruthenium in metal form include ruthenium black.

In some embodiments, ruthenium is in an oxidation state of 0, II or III in a ruthenium catalyst. In some embodiments, the ruthenium catalyst comprises no more than 20 wt %, or no more than 15 wt %, or no more than 10 wt %, or no more than 5 wt %, or no more than 1 wt %, or no more than 0.2 wt %, or no more than 0.1 wt %, or no more than 0.05 wt %, or no more than 0.02 wt %, or no more than 0.01 wt % of a ruthenium compound having oxidation state of IV or higher based on the total weight of the ruthenium catalyst. In some embodiments, the ruthenium catalyst is substantially free or free of a ruthenium compound having oxidation state of IV or higher.

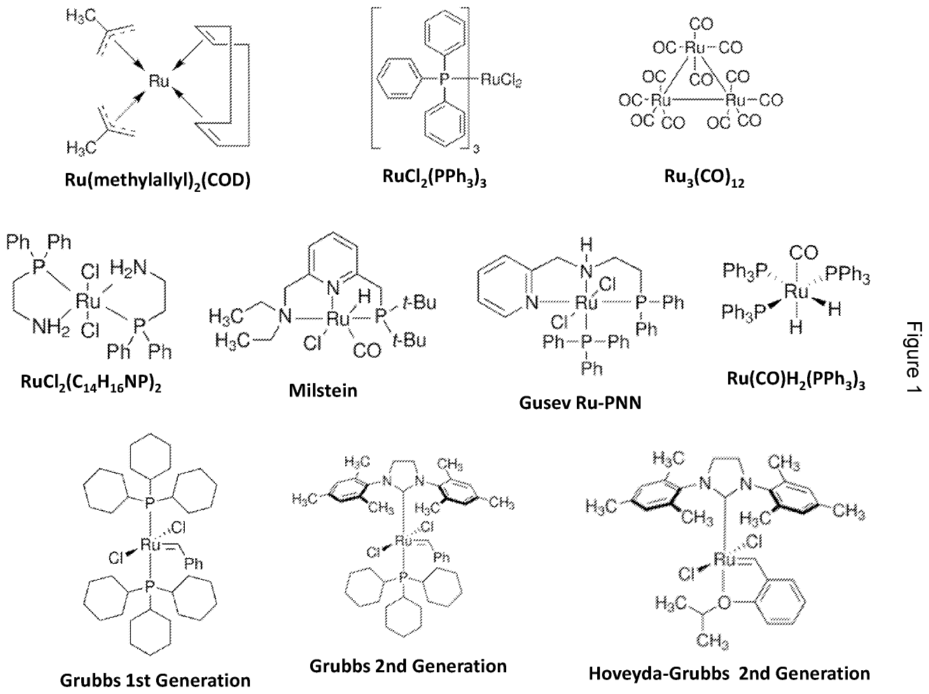

In some embodiments, the ruthenium complex is selected from the group consisting of bis(2-methylallyl)(1,5-cyclooctadiene)ruthenium(II) complex (Ru(methylallyl)₂(COD)), dichlorotris(triphenylphosphine)ruthenium(II) complex (RuCl₂(PPh₃)₃), dichlorobis(₂-(diphenylphosphino)ethylamine)ruthenium(II) complex (RuCl₂(C₁₄H₁₆NP)₂), carbonyldihydridotris(triphenylphosphine)ruthenium(II) complex (Ru(CO)H₂(PPh₃)₃), triruthenium dodecacarbonyl complex (Ru₃(CO)₁₂), dichloro(benzylidene)bis(tricyclohexylphosphine)ruthenium(II) complex (Grubbs 1^{st} generation), dichloro[1,3-bis(2,4,6-trimethylphenyl)-2-imidazolidinylidene](benzylidene) (tricyclohexylphosphine)ruthenium(II) complex (Grubbs 2^{nd} generation), dichloro[1,3-bis(2,4,6-trimethylphenyl)-2-imidazolidinylidene](2-isopropoxyphenylmethylene)ruthenium(II) complex (Hoveyda-Grubbs 2^{nd} generation), [2-(di-tert-butylphosphinomethyl)-6-(diethylaminomethyl)pyridine]carbonylchlorohydridoruthenium(II) complex (C₂₀H₃₆CIN₂OPRu, Milstein), dichlorotriphenylphosphine[2-(diphenylphosphino)-N-(2 pyridinylmethyl)ethanamine]ruthenium(II) complex (C₃₈H₃₆Cl₂N₂P₂Ru, Gusev Ru-PNN), and mixtures thereof. Molecular structures of some ruthenium complexes are shown in FIG. 1.

In some embodiments, the ruthenium complex is selected from the group consisting of bis(2-methylallyl)(1,5-cyclooctadiene)ruthenium(II) complex (Ru(methylallyl)₂(COD)), dichlorotris(triphenylphosphine)ruthenium(II) complex (RuCl₂(PPh₃)₃), and mixtures thereof. In some embodiments, the ruthenium complex is dichlorotris(triphenylphosphine)ruthenium(II) complex.

In some embodiments, the ruthenium complex is fully dissolved in the terminal alkene starting material under the process or reaction conditions in this disclosure. In some embodiments, at least 70 wt %, or at least 80 wt %, or at least 85 wt %, or at least 90 wt %, or at least 95 wt %, or at least 98 wt % of the ruthenium complex used in the process (based on the total weight of the ruthenium complex used in the process) is dissolved in the terminal alkene starting material under the process or reaction conditions.

In some embodiments, the ruthenium salt is RuCl₃ (anhydrous and/or hydrated). In some embodiments, the ruthenium salt is essentially insoluble in the terminal alkene starting material under the process or reaction conditions in this disclosure. In some embodiments, the solubility of the ruthenium salt is no more than 30 wt %, or no more than 20 wt %, or no more than 15 wt %, or no more than 10 wt %, or no more than 5 wt %, or no more than 2 wt %, or no more than 1 wt %, or no more than 0.5 wt %, or no more than 0.2 wt %, or no more than 0.1 wt %, or no more than 0.05 wt %, or no more than 0.02 wt %, or no more than 0.01 wt % (based on the total weight of the ruthenium salt used in the process) in the terminal alkene starting material under the process or reaction conditions.

In some embodiments, the amount of the ruthenium catalyst is at least 0.0001 mol %, or at least 0.0002 mol %, or at least 0.0005 mol %, or at least 0.001 mol %, or at least 0.002 mol %, or at least 0.005 mol %, or at least 0.01 mol %, or at least 0.02 mol %, or at least 0.05 mol % based on the total molar amount of the terminal alkene. In some embodiments, the amount of the ruthenium catalyst is no more than 1 mol %, or no more than 0.8 mol %, or no more than 0.5 mol %, or no more than 0.4 mol %, or no more than 0.3 mol %, or no more than 0.2 mol %, or no more than 0.15 mol %, or no more than 0.1 mol % based on the total molar amount of the terminal alkene.

In some embodiments, the ruthenium catalyst is a ruthenium complex and the amount of the ruthenium catalyst is at least 0.0001 mol %, or at least 0.0002 mol %, or at least 0.0005 mol %, or at least 0.001 mol %, or at least 0.002 mol %, or at least 0.005 mol %, or at least 0.01 mol %, or at least 0.02 mol %, or at least 0.05 mol % based on the total molar amount of the terminal alkene. In some embodiments, the ruthenium catalyst is a ruthenium complex and the amount of the ruthenium catalyst is no more than 0.2 mol %, or no more than 0.15 mol %, or no more than 0.1 mol % based on the total molar amount of the terminal alkene. In some embodiments, the amount of the ruthenium complex is from about 0.0001 mol % to about 0.2 mol %, or from about 0.001 mol % to about 0.2 mol %, or from about 0.005 mol % to about 0.2 mol %, or from about 0.01 mol % to about 0.15 mol %, or from about 0.05 mol % to about 0.1 mol % based on the total molar amount of the terminal alkene.

In some embodiments, the ruthenium catalyst is a ruthenium salt and the amount of the ruthenium catalyst is at least 0.01 mol %, or at least 0.02 mol %, or at least 0.05 mol % based on the total molar amount of the terminal alkene. In some embodiments, the ruthenium catalyst is a ruthenium salt and the amount of the ruthenium catalyst is no more than 1 mol %, or no more than 0.8 mol %, or no more than 0.5 mol %, or no more than 0.4 mol %, or no more than 0.3 mol %, or no more than 0.2 mol % based on the total molar amount of the terminal alkene. In some embodiments, the amount of the ruthenium salt is from about 0.01 mol % to about 1 mol %, or from about 0.01 mol % to about 0.5 mol %, or from about 0.02 mol % to about 0.3 mol %, or from about 0.05 mol % to about 0.2 mol % based on the total molar amount of the terminal alkene.

In some embodiments, the ruthenium catalyst comprises ruthenium or ruthenium compound supported on a catalyst support or carrier, that is, the ruthenium catalyst is a supported ruthenium catalyst. In some embodiments, the catalyst support is selected from the group consisting of silica, alumina, carbon (e.g., activated carbon), TiO₂, zeolite, and mixtures thereof. In some embodiments, the catalyst support is activated to provide more surface area. The catalyst support can be in any convenient form including particles, powders, granules, fibers, or shaped pieces.

Ruthenium supported on the catalyst support can be in a cationic form (e.g., Ru⁺², Ru⁺³) or in a metal form. In some embodiments, the ruthenium catalyst comprises RuCl₃ supported on a catalyst support. In some embodiments, the ruthenium catalyst comprises ruthenium nanoparticles supported on a catalyst support. The supported ruthenium catalyst can be made by means known in the art including precipitation, coprecipitation, impregnation and methods of deposition or combination known in the art.

In some embodiments, the amount of ruthenium supported on a catalyst support is at least 0.01 wt %, or at least 0.02 wt %, or at least 0.05 wt %, or at least 0.1 wt %, or at least 0.2 wt %, or at least 0.5 wt %, or at least 1 wt % based on the total weight of the supported ruthenium catalyst. In some embodiments, the amount of ruthenium supported on a catalyst support is no more than 30 wt %, or no more than 25 wt %, or no more than 20 wt %, or no more than 15 wt %, or no more than 10 wt %, or no more than 5 wt % based on the total weight of the supported ruthenium catalyst. In embodiments of the supported ruthenium catalyst, the amount of ruthenium (supported on a catalyst support) means the amount of ruthenium element.

In some embodiments, the ruthenium catalyst comprises ruthenium or ruthenium compound supported on a catalyst support and the amount of ruthenium (supported on the catalyst support) is at least 0.0001 mol %, or at least 0.0002 mol %, or at least 0.0005 mol %, or at least 0.001 mol %, or at least 0.002 mol %, or at least 0.005 mol %, or at least 0.01 mol % comparing with the total molar amount of the terminal alkene. In some embodiments, the ruthenium catalyst comprises ruthenium or ruthenium compound supported on a catalyst support and the amount of ruthenium (supported on the catalyst support) is no more than 1 mol %, or no more than 0.5 mol %, or no more than 0.2 mol %, or no more than 0.1 mol % comparing with the total molar amount of the terminal alkene.

In the process of this disclosure, the isomerizing process is conducted at a temperature of at least about 120° C., or at least about 130° C., or at least about 140° C. In some embodiments, the isomerizing process is conducted at a temperature of no more than about 250° C., or no more than about 240° C., or no more than about 230° C., or no more than about 220° C., or no more than about 210° C., or no more than about 200° C. In some embodiments, the temperature is in a range of from about 120° C. to about 250° C., or from about 120° C. to about 240° C., or from about 120° C. to about 220° C., or from about 120° C. to about 200° C., or from about 130° C. to about 250° C., or from about 130° C. to about 240° C., or from about 130° C. to about 220° C., or from about 140° C. to about 250° C., or from about 140° C. to about 220° C.

The isomerizing process in this disclosure can be carried out under atmospheric pressure or under pressures less than or greater than atmospheric pressure. For example, the process may be carried out at a pressure ranging from about 30 millibar to about 5 bar. In some embodiments, the isomerizing process is carried out under atmospheric pressure.

The isomerizing process in this disclosure can be conducted under ambient atmosphere (i.e., air) or inert atmosphere. In some embodiments, the isomerizing process is conducted under inert atmosphere such as under an inert gas atmosphere. Examples of inert gases include nitrogen and noble gases such as argon. In some embodiments, the isomerizing process is conducted under a nitrogen gas atmosphere. In practice, the inert atmosphere may still contain minor amounts of oxygen. In some embodiments, the isomerizing process is conducted under an inert gas atmosphere and under a pressure greater than atmospheric pressure. In some embodiments, the isomerizing process is conducted under ambient atmosphere.

In some embodiments, the isomerizing process time or isomerization reaction time is at least 10 minutes, or at least 20 minutes, or at least 0.5 hour, or at least 1 hour, or at least 1.5 hours, or at least 2 hours. In some embodiments, the isomerizing process time or isomerization reaction time is no more than 72 hours, or no more than 50 hours, or no more than 30 hours, or no more than 20 hours, or no more than 15 hours, or no more than 10 hours, or no more than 8 hours. In some embodiments, the isomerizing process time or isomerization reaction time is in a range of from about 0.5 to about 72 hours, or in a range of from about 1 to about 30 hours, or in a range of from about 2 to about 10 hours.

In some embodiments, the isomerizing process is conducted in the presence of a solvent. Examples of the solvent include alcohols, ethers, pentane, hexane, methylene chloride, chloroform and ethyl acetate. Examples of alcohol include methanol, ethanol, 1-propanol, isopropanol, butanol and its isomers, and pentanol and its isomers. In some embodiments, alcohol is a tertiary alcohol such as tert-amyl alcohol. In some embodiments, the amount of the solvent present in the reaction zone during the reaction is no more than 50 wt %, or no more than 40 wt %, or no more than 30 wt %, or no more than 20 wt %, or no more than 10 wt %, or no more than 5 wt %, or no more than 2 wt %, or no more than 1 wt %, or no more than 0.5 wt % based on the total weight of the terminal alkene. In some embodiments, the reaction zone is substantially free or free of a solvent, that is, the isomerizing process is conducted essentially in the absence of or in the absence of a solvent.

In some embodiments, the reaction zone is substantially free or free of an additive, that is, the isomerizing process is conducted essentially in the absence of or in the absence of an additive. Typical additives include bases (organic bases or inorganic bases) such as amines and other nitrogen-containing organic bases, acetates, hydroxides and tert-butoxides, and co-catalysts such as strong Lewis acids (e.g., BF₃) and strong Bronsted acids (e.g., triflic acid). In some embodiments, the total amount of the additives present in the reaction zone during the reaction is no more than 0.1 wt %, or no more than 0.05 wt %, or no more than 0.01 wt %, or no more than 0.005 wt %, or no more than 0.001 wt %, or no more than 0.0005 wt %, or no more than 0.0001 wt % based on the total weight of the terminal alkene.

In some embodiments, the reaction zone is substantially free or free of an additional ligand, that is, the isomerizing process is conducted essentially in the absence of or in the absence of an additional ligand. By “additional ligand” means a ligand which is not present in the ruthenium complex used for the reaction. Typical additional ligands include carbon monoxide, carbene, phosphine and alkene-based compounds. In some embodiments, the total amount of the additional ligands present in the reaction zone during the reaction is no more than 0.1 wt %, or no more than 0.05 wt %, or no more than 0.01 wt %, or no more than 0.005 wt %, or no more than 0.001 wt %, or no more than 0.0005 wt %, or no more than 0.0001 wt % based on the total weight of the terminal alkene. In some embodiments, essentially no additional ligand is fed into the reaction zone before or during the reaction.

In some embodiments, the reaction zone is substantially free or free of an acid, that is, the isomerizing process is conducted essentially in the absence of or in the absence of an acid. Typical acids include triflic acid, HBF4 and sulfuric acid. In some embodiments, the total amount of the acids present in the reaction zone during the reaction is no more than 0.1 wt %, or no more than 0.05 wt %, or no more than 0.01 wt %, or no more than 0.005 wt %, or no more than 0.001 wt %, or no more than 0.0005 wt %, or no more than 0.0001 wt % based on the total weight of the terminal alkene.

In some embodiments, the process in this disclosure comprises feeding a terminal alkene and a ruthenium catalyst into a reaction zone, and isomerizing the terminal alkene to form a product comprising an internal alkene in the presence of the ruthenium catalyst at a temperature of at least about 120° C., wherein the terminal alkene and the ruthenium catalyst are sole chemical reagents fed into the reaction zone before and during the isomerization reaction, that is, no chemical reagents other than the terminal alkene and the ruthenium catalyst is fed into the reaction zone before and during the isomerization reaction. It is to be appreciated that the terminal alkene and the ruthenium catalyst may comprise impurities respectively.

One advantage of the process in this disclosure is that it generates little or no HCl. When a chlorine-containing ruthenium catalyst such as RuCl₃ is used in the process, a small amount of HCl may be generated. Typically during the process of this disclosure, no more than 3 mol %, or no more than 2 mol %, or no more than 1 mol %, or no more than 0.5 mol %, or no more than 0.1 mol %, or no more than 0.05 mol %, or no more than 0.01 mol %, or no more than 0.005 mol %, or no more than 0.001 mol % of HCl is generated based on the total molar amount of the terminal alkene. In some embodiments, essentially no or no HCl is generated during the process.

During the process of this disclosure, the terminal alkene is isomerized to form the internal alkene. It has been found that a double bond can migrate along a linear (unbranched) hydrocarbon chain during the process of this disclosure as shown in Scheme 1.

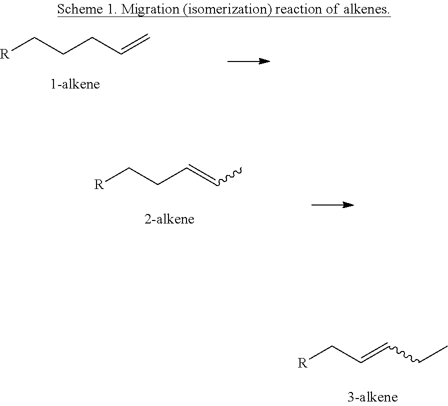

It has also been found that such double bond migration (along the hydrocarbon chain) stops at a branched carbon. It has further been found that the process of this disclosure does not work when a secondary alkene, a tertiary alkene, or a quaternary alkene is used as the starting material, that is, the process of this disclosure is selective for a primary alkene starting material.

In some embodiments, the internal alkene product comprises an organic moiety represented by Formula II set forth below, that is, the double bond only migrates internally one position.

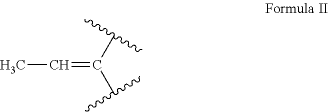 [SMILES: *C(*)=CC]

In some embodiments, the internal alkene product comprises an organic moiety represented by Formula IV or Formula V set forth below, that is, the double bond migrates internally one or more positions.

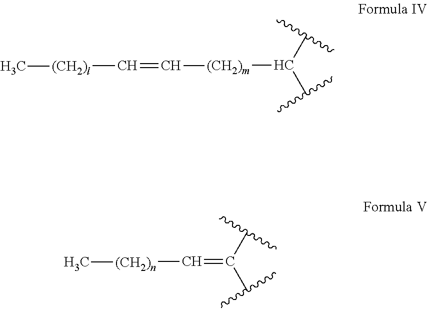

In Formulas IV and V, n is an integer from 1 to 20. In some embodiments, n is an integer from 1 to 15, or an integer from 1 to 10. In some embodiments, n is 1. Moreover, I and m independently is an integer of 0 or greater than 0 (positive integer), and I+m=n−1.

The isomerization reaction in this disclosure has high conversion, selectivity and yield. In some embodiments, the yield of the internal alkene (i.e., isomerization reaction yield) is at least 70%, or at least 80%, or at least 85%, or at least 90%, or at least 95%. In some embodiments, the yield of the internal alkene is up to 98%, or up to 98.5%, or up to 99%, or up to 99.5%, or up to 100%. In some embodiments, the yield of the internal alkene is in a range of from 70% to 100%, or in a range of from 90% to 99%, or in a range of from 95% to 98.5%.

In some embodiments, the process of this disclosure also comprises recovering the internal alkene. The internal alkene product can be recovered using procedures well known to the art. In some embodiments, the internal alkene is recovered by distillation (e.g., fractional distillation). In some embodiments, the internal alkene product can be used as fragrance ingredients or intermediates for the synthesis of fragrance ingredients.

In some embodiments, the terminal alkene starting material is a mixture of 2-propoxy-5-vinylcyclohexan-1-ol and 2-propoxy-4-vinylcyclohexan-1-ol, and the internal alkene product is a mixture of 2-propoxy-5-ethylidenecyclohexan-1-ol and 2-propoxy-4-ethylidenecyclohexan-1-ol. During the isomerizing process, 2-propoxy-5-vinylcyclohexan-1-ol is converted to 2-propoxy-5-ethylidenecyclohexan-1-ol, and 2-propoxy-4-vinylcyclohexan-1-ol is converted to 2-propoxy-4-ethylidenecyclohexan-1-ol. In some embodiments, the ruthenium catalyst is a ruthenium complex and the amount of the ruthenium catalyst is at least 0.001 mol %, or at least 0.002 mol %, or at least 0.005 mol %, or at least 0.01 mol %, or at least 0.02 mol % based on the total molar amount of the terminal alkene. In some embodiments, the ruthenium catalyst is a ruthenium complex and the amount of the ruthenium catalyst is no more than 0.2 mol %, or no more than 0.15 mol %, or no more than 0.1 mol % based on the total molar amount of the terminal alkene. In some embodiments, the amount of the ruthenium complex is from about 0.001 mol % to about 0.2 mol %, or from about 0.005 mol % to about 0.15 mol %, or from about 0.01 mol % to about 0.1 mol % based on the total molar amount of the terminal alkene.

In some embodiments, the terminal alkene starting material is methyl eugenol (1,2-dimethoxy-4-(prop-2-en-1-yl)benzene) and the internal alkene product is methyl isoeugenol (1,2-dimethoxy-4-(prop-1-en-1-yl)benzene). The term “methyl isoeugenol”, as used herein, includes both cis and trans isomers. In some embodiments, the ruthenium catalyst is a ruthenium complex and the amount of the ruthenium catalyst is at least 0.0001 mol %, or at least 0.0002 mol %, or at least 0.0005 mol %, or at least 0.001 mol % based on the total molar amount of the terminal alkene. In some embodiments, the ruthenium catalyst is a ruthenium complex and the amount of the ruthenium catalyst is no more than 0.2 mol %, or no more than 0.15 mol %, or no more than 0.1 mol %, or no more than 0.05 mol %, or no more than 0.01 mol % based on the total molar amount of the terminal alkene. In some embodiments, the amount of the ruthenium complex is from about 0.0001 mol % to about 0.05 mol %, or from about 0.0005 mol % to about 0.01 mol % based on the total molar amount of the terminal alkene.

In some embodiments, the terminal alkene starting material is 9-decen-1-ol and the internal alkene product comprises a mixture of 6-decen-1-ol, 7-decen-1-ol and 8-decen-1-ol. In some embodiments, the internal alkene product further comprises other position isomers such as 5-decen-1-ol and 4-decen-1-ol. In some embodiments, the ruthenium catalyst is a ruthenium complex and the amount of the ruthenium catalyst is at least 0.001 mol %, or at least 0.002 mol %, or at least 0.005 mol %, or at least 0.01 mol %, or at least 0.02 mol % based on the total molar amount of the terminal alkene. In some embodiments, the ruthenium catalyst is a ruthenium complex and the amount of the ruthenium catalyst is no more than 0.2 mol %, or no more than 0.15 mol %, or no more than 0.1 mol % based on the total molar amount of the terminal alkene. In some embodiments, the amount of the ruthenium complex is from about 0.001 mol % to about 0.2 mol %, or from about 0.005 mol % to about 0.15 mol %, or from about 0.01 mol % to about 0.1 mol % based on the total molar amount of the terminal alkene.

Many aspects and embodiments have been described above and are merely exemplary and not limiting. After reading this specification, skilled artisans appreciate that other aspects and embodiments are possible without departing from the scope of the invention.

### EXAMPLES

The concepts described herein will be further described in the following examples, which do not limit the scope of the invention described in the claims.

### General

Glassware was dried in an oven at 175° C. before use. Reactions were performed in 2 mL or 8 mL vials equipped with a magnetic stirrer and closed with a steel cap having a rubber septum part to sample out. Reagents and solvents were obtained from commercial sources and were used without further purification unless otherwise indicated. Products were characterised by GC-MS, ¹H- and ¹³C-NMR, and DEPT (distortionless enhancement by polarization transfer). Gas chromatographic analyses were performed in an instrument equipped with a 25 m capillary column of 5% phenylmethylsilicone. N-dodecane was used as an external standard. GC/MS analyses were performed on a spectrometer equipped with the same column as the GC and operated under the same conditions. ¹H, ¹³C and DEPT measurements were recorded in a 300 MHz instrument using CDCI₃ as a solvent, containing TMS as an internal standard.

### Example 1: Synthesis of Veraspice With Different Amounts of Ru(Methylallyl)₂(COD)

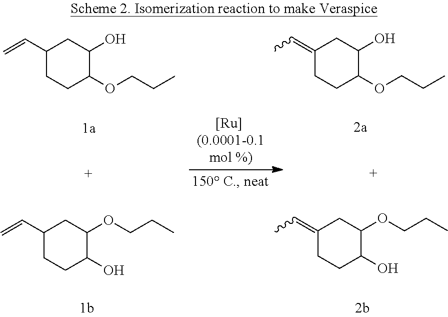

A mixture of two regioisomers 2-propoxy-5-vinylcyclohexan-1-ol (1a) and 2-propoxy-4-vinylcyclohexan-1-ol (1b) in 1-propanol solvent was concentrated under reduced pressure to remove the solvent. Then, the mixture (0.2-2 g) was charged into a 2 mL or 8 mL vial equipped with a magnetic stirrer and the Ru(methylallyl)₂(COD) catalyst (0.0001-0.1 mol %) was added. The vial was closed with a cap, placed in a pre-heated bath oil at the reaction temperature of 150° C. under magnetically stirring and maintained during the reaction time. The product mixture comprising 2-propoxy-5-ethylidenecyclohexan-1-ol (2a) and 2-propoxy-4-ethylidenecyclohexan-1-ol (2b) was characterised by GC and NMR. Results are shown in Table 1.

TABLE 1

| | Catalyst Amount | Reaction Time | Yield |
|---|---|---|---|
| Entry | (mol %) | (hour) | (%) |
| 1 | 0.1 | 1 | 97.8 |
| 2 | 0.05 | 2.5 | 98.2 |
| 3 | 0.01 | 21 | 89.5 |
| 4 | 0.005 | 21 | 70.5 |
| 5 | 0.001 | 64 | 25.0 |
| 6 | 0.0005 | 64 | 15.1 |
| 7 | 0.0001 | 64 | 4.1 |

The isomerization reactions were carried out with the terminal alkene starting material and the ruthenium catalyst alone, that is, the isomerization reactions were carried out essentially free of a solvent, an additive, an acid, and an additional ligand. This example demonstrated that Veraspice fragrance ingredients 2a and 2b can be efficiently made with high yield by using Ru(methylallyl)₂(COD) as the catalyst at very low concentration in the absence of a solvent.

### Example 2: Synthesis of Veraspice With Different Amounts of RuCl₂(PPh₃)₃

Same process as Example 1 was conducted in Example 2 except that RuCl₂(PPh₃)₃ was used as the catalyst for Example 2. The reaction temperature is also 150° C. Results are shown in Table 2.

TABLE 2

| | Catalyst Amount | Reaction Time | Yield |
|---|---|---|---|
| Entry | (mol %) | (hour) | (%) |
| 1 | 0.1 | 1 | 98.0 |
| 2 | 0.08 | 5 | 98.0 |
| 3 | 0.05 | 23 | 98.1 |
| 4 | 0.05 | 22 | 94.8 |
| 5 | 0.01 | 22 | 92.5 |
| 6 | 0.005 | 22 | 72.6 |
| 7 | 0.001 | 22 | 22.7 |
| 8 | 0.0005 | 22 | 12.2 |
| 9 | 0.0001 | 22 | 3.0 |

The isomerization reactions were carried out with the terminal alkene starting material and the ruthenium catalyst alone, that is, the isomerization reactions were carried out essentially free of a solvent, an additive, an acid, and an additional ligand. This example demonstrated that Veraspice fragrance ingredients 2a and 2b can be efficiently made with high yield by using RuCl₂(PPh₃)₃ as the catalyst at very low concentration in the absence of a solvent.

### Example 3: Synthesis of Veraspice With Different Amounts of RuCl₃

Same process as Example 1 was conducted in Example 3 except that RuCl₃ was used as the catalyst for Example 3. The reaction temperature is also 150° C. Results are shown in Table 3.

TABLE 3

| | Catalyst Amount | Reaction Time | Yield |
|---|---|---|---|
| Entry | (mol %) | (hour) | (%) |
| 1 | 0.2 | 6 | 88.8 |
| 2 | 0.2 | 6 | 97.0 |
| 3 | 0.13 | 6 | 97.9 |
| 4 | 0.1 | 6 | 97.7 |
| 5 | 0.05 | 6 | 98.4 |
| 6 | 0.01 | 22 | 49.6 |

The isomerization reactions were carried out with the terminal alkene starting material and the ruthenium catalyst alone, that is, the isomerization reactions were carried out essentially free of a solvent, an additive, an acid, and an additional ligand. This example demonstrated that Veraspice fragrance ingredients 2a and 2b can be efficiently made with high yield by using RuCl₃ as the catalyst at low concentration in the absence of a solvent.

### Example 4: Synthesis of Methyl Isoeugenol With Different Amounts of Ru(Methylallyl)₂(COD)

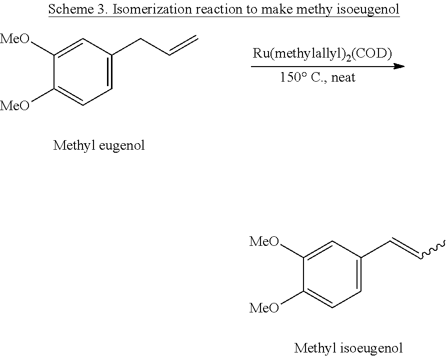

Methyl eugenol (1 mL) was charged into a 2 mL or 8 mL vial equipped with a magnetic stirrer and the Ru(methylallyl)₂(COD) catalyst (0.0001-5 mol %) was added. The vial was closed with a cap, placed in a pre-heated bath oil at the reaction temperature of 150° C. under magnetically stirring and maintained during the reaction time. The product mixture comprising methyl isoeugenol was characterised by GC and NMR. Results are shown in Table 4.

TABLE 4

| | Catalyst Amount | Reaction | | Yield |
|---|---|---|---|---|
| Entry | (mol %) | Time | | (%) |
| 1 | 5 | 1 | minute | 100 |
| 2 | 1 | 1 | minute | 100 |
| 3 | 0.005 | 4 | hours | 90 |
| 4 | 0.001 | 7 | hours | 78 |
| 5 | | 22 | hours | 96 |
| 6 | 0.0005 | 21 | hours | 94 |
| 7 | 0.0001 | 20 | hours | 81 |

The isomerization reactions were carried out with the terminal alkene starting material and the ruthenium catalyst alone, that is, the isomerization reactions were carried out essentially free of a solvent, an additive, an acid, and an additional ligand. This example demonstrated that methyl isoeugenol can be efficiently made with high yield by using Ru(methylallyl)₂(COD) as the catalyst at very low concentration in the absence of a solvent.

### Example 5: Synthesis of Methyl Isoeugenol With Different Catalysts

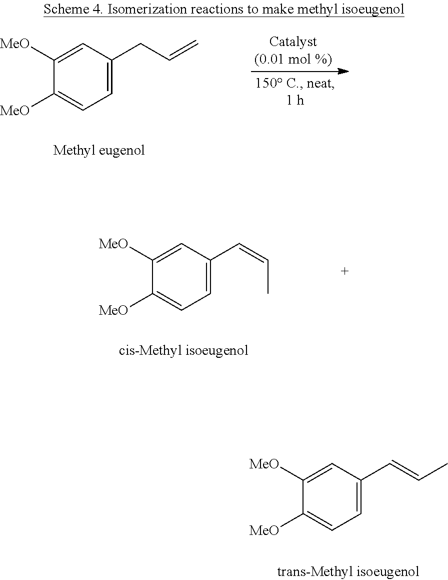

Methyl eugenol (1 mL) was charged into a 2 mL or 8 mL vial equipped with a magnetic stirrer and a catalyst (0.01 mol %) was added. The vial was closed with a cap, placed in a pre-heated bath oil at the reaction temperature of 150° C. under magnetically stirring and maintained for one hour (reaction time). After one hour reaction time, the reaction was stopped and cooled. The product mixture was characterised by GC and NMR for the cis and trans methyl isoeugenol content. Results are shown in Table 5.

TABLE 5

| | | Conversion | Yield (%) | |
|---|---|---|---|---|
| Entry | Catalyst | (%) | cis | trans |
| 1 | FeCl₂ | 0.0 | 0.0 | 0.0 |
| 2 | Fe(CO)₅ dis. | 0.1 | 0.0 | 0.0 |
| 3 | Ferrocene | 0.0 | 0.0 | 0.0 |
| 4 | Co(NO₃)₂•6H₂O | 0.0 | 0.0 | 0.0 |
| 5 | Ni(NO₃)₂•6H₂O | 0.0 | 0.0 | 0.0 |
| 6 | NiCl₂glyme | 0.0 | 0.0 | 0.0 |
| 7 | Cu(NO₃)₂•3H₂O | 0.0 | 0.0 | 0.0 |
| 8 | [(iPr)CuCl] | 0.0 | 0.0 | 0.0 |
| 9 | Na₂PdCl₄•3H₂O | 30.7 | 3.4 | 27.3 |
| 10 | RhCl₃•xH₂O | 61.7 | 17.5 | 44.3 |
| 11 | [Rh(COD)Cl]₂ | 23.5 | 4.8 | 18.7 |
| 12 | IrCl₃•xH₂O | 17.0 | 2.6 | 14.4 |
| 13 | [Ir(COD) Cl]₂ | 28.6 | 3.6 | 25.0 |
| 14 | RuCl₂(PPh₃)₃ | 99.5 | 8.9 | 90.7 |
| 15 | Ru(methylallyl)₂(COD) | 98.0 | 13.1 | 85.0 |

In Table 5, “Fe(CO)₅ dis.” means Fe(CO)₅ previously dissolved in methyl eugenol before adding to the vial. In this experiment, the total amount of methyl eugenol fed to the vial was still 1 mL.

The isomerization reactions were carried out with the terminal alkene starting material and the catalyst alone, that is, the isomerization reactions were carried out essentially free of a solvent, an additive, an acid, and an additional ligand. This example demonstrated that while the ruthenium catalyst got nearly quantitatively the isomerized product in just one hour reaction time, with very high selectivity and without branched nor oligomerized products, other metal catalysts were barely active under solvent- and additive-free reaction conditons.

### Example 6: Synthesis of Methyl Isoeugenol With Different Ruthenium Catalysts

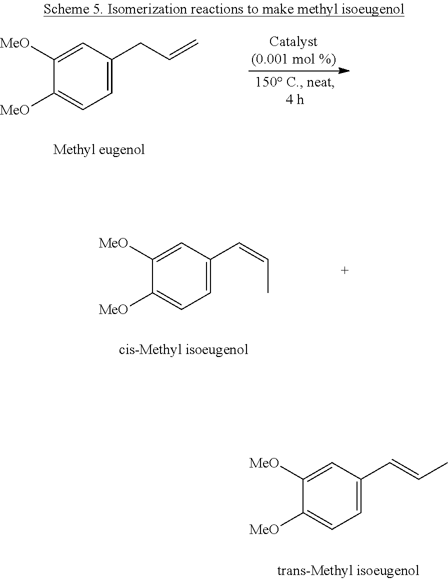

Methyl eugenol (1 mL) was charged into a 2 mL or 8 mL vial equipped with a magnetic stirrer and a ruthenium catalyst (0.001 mol %) was added. The vial was closed with a cap, placed in a pre-heated bath oil at the reaction temperature of 150° C. under magnetically stirring and maintained for four hours (reaction time). After four hours reaction time, the reaction was stopped and cooled. The product mixture comprising methyl isoeugenol was characterised by GC and NMR. Results are shown in Table 6.

TABLE 6

| | | Conversion | Yield (%) | |
|---|---|---|---|---|
| Entry | Catalyst | (%) | cis | trans |
| 1 | RuO₂ | 0.0 | 0.0 | 0.0 |
| 2 | Ru(methylallyl)₂(COD) | 83.9 | 16.5 | 67.4 |
| 3 | RuCl₂(PPh₃)₃ | 95.0 | 16.6 | 78.3 |
| 4 | RuCl₂(C₁₄H₁₆NP)₂ | 93.7 | 17.0 | 76.8 |
| 5 | Milstein | 72.5 | 15.8 | 56.6 |
| 6 | Gusev Ru—PNN | 75.7 | 16.4 | 59.2 |
| 7 | Ru(CO)H₂(PPh₃)₃ | 98.3 | 14.9 | 83.3 |
| 8 | Ru₃(CO)₁₂ | 99.4 | 9.2 | 90.2 |
| 9 | Grubbs 1^{st} | 99.3 | 12.6 | 86.7 |
| | Generation | | | |
| 10 | Grubbs 2^{nd} | 97.9 | 15.3 | 82.6 |
| | Generation | | | |
| 11 | Hoveyda-Grubbs 2^{nd} | 98.8 | 14.0 | 84.8 |
| | Generation | | | |

The isomerization reactions were carried out with the terminal alkene starting material and the ruthenium catalyst alone, that is, the isomerization reactions were carried out essentially free of a solvent, an additive, an acid, and an additional ligand. This example demonstrated that methyl isoeugenol can be efficiently made with high yield by using ruthenium complexes at 0.001 mol% (10 ppm) amount in the absence of a solvent.

### Example 7: Synthesis of Methyl Isoeugenol With Different Supported Ruthenium Catalysts

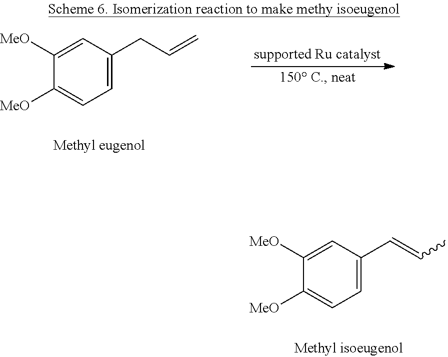

Methyl eugenol (1 mL) was charged into a 2 mL or 8 mL vial equipped with a magnetic stirrer and a ruthenium catalyst comprising ruthenium supported on a catalyst support (supported ruthenium catalyst) was added. The vial was closed with a cap, placed in a pre-heated bath oil at the reaction temperature of 150° C. under magnetically stirring and maintained during the reaction time. The supported ruthenium catalysts were either commercial or prepared by impregnation of aqueous RuCl₃. The product mixture comprising methyl isoeugenol was characterised by GC and NMR. Results are shown in Table 7.

TABLE 7

| | | Catalyst Amount | Reaction Time | Yield |
|---|---|---|---|---|
| Entry | Catalyst | (mol % Ru) | (hour) | (%) |
| 1 | Ru on silica | 0.005 | 1 | 99.0 |
| | (5 wt % Ru) | | | |
| 2 | Ru on TiO₂ | 0.1 | 17 | 97.7 |
| | (5 wt % Ru) | | | |
| 3 | Ru on alumina | 0.1 | 17 | 89.3 |
| | (3 wt % Ru) | | | |
| 4 | Ru on alumina | 0.2 | 20 | 88.2 |
| | (5 wt % Ru) | | | |
| 5 | Ru on carbon | 0.1 | 18 | 81.0 |
| | (5 wt % Ru) | | | |
| 6 | Ru on KY | 0.01 | 22 | 95.3 |
| | (1 wt % Ru) | | | |

In Table 7, “wt % Ru” means the amount of ruthenium supported on the catalyst support based on the total weight of the supported ruthenium catalyst, “mol % Ru” means the amount of ruthenium supported on the catalyst support comparing with the total molar amount of the terminal alkene, and KY means zeolite Y with potassium.

The isomerization reactions were carried out with the terminal alkene starting material and the supported ruthenium catalyst alone, that is, the isomerization reactions were carried out essentially free of a solvent, an additive, an acid, and an additional ligand. This example demonstrated that methyl isoeugenol can be efficiently made with high yield by using supported ruthenium catalysts at very low ruthenium concentration in the absence of a solvent.

### Example 8: Synthesis of Isorosalva With Ru(Methylallyl)₂(COD)

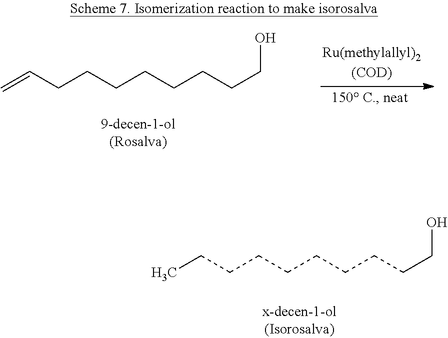

In Scheme 7, the broken line represents one double bond and six single bonds.

9-decen-1-ol (1 mL) was charged into a 2 mL or 8 mL vial equipped with a magnetic stirrer and the Ru(methylallyl)₂(COD) catalyst (0.0001-0.1 mol %) was added. The vial was closed with a cap, placed in a pre-heated bath oil at the reaction temperature of 150° C. under magnetically stirring and maintained during the reaction time. The product mixture comprising isomers x-decen-1-ol (x is an integer from 2 to 8) was characterised by GC and NMR. Results are shown in Table 8.

TABLE 8

| | Cat. | | | GC Peak Area of x-decen-1-ol Isomers | | | | | | |
|---|---|---|---|---|---|---|---|---|---|---|
| En- | (mol | T | Yield | x = | | | | | | x = |
| try | %) | (h) | (%) | 2 | x = 3 | x = 4 | x = 5 | x = 6 | x = 7 | 8 |
| 1 | 0.1 | 1 | 97 | 2.2 | 5.8 | 27.6 | 27.6 | 23.3 | 9.75 | 33.0 |
| 2 | 0.05 | 2 | 97 | 2.0 | 3.8 | 21.9 | 30.4 | 27.0 | 11.1 | 38.1 |
| 3 | 0.01 | 3 | 96 | 1.0 | 3.3 | 21.8 | 28.5 | 29.5 | 14.2 | 43.7 |
| 4 | 0.005 | 4 | 96 | 0.8 | 1.9 | 18.0 | 27.5 | 34.1 | 16.4 | 50.5 |
| 5 | 0.001 | 22 | 95 | 0.5 | 0.2 | 8.2 | 28.2 | 44.6 | 18.3 | 62.9 |
| 6 | 0.0005 | 22 | 95 | 0.5 | 0.0 | 5.0 | 26.0 | 47.5 | 21.0 | 68.5 |
| 7 | 0.0001 | 22 | 91 | 0.5 | 0.0 | 8.3 | 14.3 | 51.0 | 25.8 | 76.8 |

In Tables 8, 9 and 10, “Cat. (mol %)” means the amount of Ru(methylallyl)₂(COD) catalyst in mol % based on the total molar amount of the terminal alkene, and “T (h)” means reaction time in hours.

The isomerization reactions were carried out with the terminal alkene starting material and the ruthenium catalyst alone, that is, the isomerization reactions were carried out essentially free of a solvent, an additive, an acid, and an additional ligand. This example demonstrated that isorosalva fragrance intermediates can be efficiently made with high yield by using Ru(methylallyl)₂(COD) as the catalyst at very low concentration in the absence of a solvent.

### Example 9: Synthesis of Isorosalva With Ru(Methylallyl)₂(COD)

Same process as Example 8 was conducted in Example 9 except that the reaction temperature is 175° C. in this Example. Results are shown in Table 9.

TABLE 9

| | Cat. | | | GC Peak Area of x-decen-1-ol Isomers | | | | | | |
|---|---|---|---|---|---|---|---|---|---|---|
| En- | (mol | T | Yield | x = | | | x = | x = | x = | x = |
| try | %) | (h) | (%) | 2 | x = 3 | x = 4 | 5 | 6 | 7 | 8 |
| 1 | 0.01 | 0.5 | 96 | 1.5 | 7.6 | 30.6 | 27.0 | 19.2 | 8.2 | 27.4 |
| 2 | 0.005 | 0.5 | 96 | 1.1 | 5.40 | 29.0 | 29.6 | 22.7 | 9.7 | 32.4 |
| 3 | 0.001 | 1 | 96 | 0.4 | 1.7 | 21.0 | 33.4 | 30.2 | 12.8 | 43.0 |
| 4 | 0.0005 | 1 | 95 | 0.4 | 0.8 | 15.4 | 33.7 | 35.0 | 14.7 | 49.7 |
| 5 | 0.0001 | 22 | 94 | 0.4 | 0.0 | 5.5 | 13.5 | 56 | 24.5 | 80.4 |

The isomerization reactions were carried out with the terminal alkene starting material and the ruthenium catalyst alone, that is, the isomerization reactions were carried out essentially free of a solvent, an additive, an acid, and an additional ligand. This example demonstrated that isorosalva fragrance intermediates can be efficiently made with high yield by using Ru(methylallyl)₂(COD) as the catalyst at very low concentration in the absence of a solvent. In Entry 1 where the amount of the Ru(methylallyl)₂(COD) catalyst was 0.01 mol %, about 3 mol % decanal was formed.

### Example 10: Synthesis of Isorosalva With Ru(Methylallyl)₂(COD)

Same process as Example 8 was conducted in Example 10 except that the reaction temperature is 200° C. in this Example. Results are shown in Table 10.

TABLE 10

| | Cat. | | | GC Peak Area of x-decen-1-ol Isomers | | | | | | |
|---|---|---|---|---|---|---|---|---|---|---|
| En- | (mol | | Yield | x = | x = | | | | | x = |
| try | %) | T (h) | (%) | 2 | 3 | x = 4 | x = 5 | x = 6 | x = 7 | 8 |
| 1 | 0.01 | 0.5 | 98 | 1.8 | 8.4 | 30.5 | 27.0 | 17.7 | 7.8 | 25.5 |
| 2 | 0.005 | 0.5 | 96 | 1.3 | 6.4 | 29.6 | 30.1 | 21.8 | 9.6 | 31.4 |
| 3 | 0.001 | 0.5 | 96 | 0.8 | 3.3 | 24.2 | 33.3 | 26.0 | 11.5 | 37.5 |
| 4 | 0.0005 | 1 | 96 | 0.7 | 2.2 | 21.6 | 33.6 | 28.7 | 12.8 | 41.5 |
| 5 | 0.0001 | 22 | 94 | 0.5 | 1.0 | 6.0 | 15.1 | 51.0 | 24.0 | 75.0 |

The isomerization reactions were carried out with the terminal alkene starting material and the ruthenium catalyst alone, that is, the isomerization reactions were carried out essentially free of a solvent, an additive, an acid, and an additional ligand. This example demonstrated that isorosalva fragrance intermediates can be efficiently made with high yield by using Ru(methylallyl)₂(COD) as the catalyst at very low concentration in the absence of a solvent. In Entry 1 where the amount of the Ru(methylallyl)₂(COD) catalyst was 0.01 mol %, about 6 mol % decanal was formed.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination.

1. A process for making fragrance ingredient or fragrance intermediate, comprising: isomerizing a starting material comprising a terminal alkene to form a product comprising an internal alkene in the presence of a ruthenium catalyst at a temperature of at least about 120° C.

2. The process of claim 1, wherein the internal alkene comprises an organic moiety represented by Formula II set forth below:

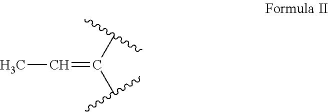 [SMILES: *C(*)=CC]

3. The process of claim 1, wherein the temperature is in a range of from about 120° C. to about 250° C.

4. The process of claim 1, wherein the ruthenium catalyst is a ruthenium complex.

5. The process of claim 4, wherein the amount of the ruthenium catalyst is in a range of from about 0.0001 mol % to about 0.2 mol % based on the total molar amount of the terminal alkene.

6. The process of claim 1, wherein the ruthenium catalyst is a ruthenium salt.

7. The process of claim 6, wherein the amount of the ruthenium catalyst is in a range of from about 0.01 mol % to about 1 mol % based on the total molar amount of the terminal alkene.

8. The process of claim 1, wherein the ruthenium catalyst is selected from the group consisting of ruthenium complexes, ruthenium salts, ruthenium in metal form, and mixtures thereof.

9. The process of claim 1, wherein the ruthenium catalyst is selected from the group consisting of bis(2-methylallyl)(1,5-cyclooctadiene)ruthenium(II) complex (Ru(methylallyl)₂(COD)), dichlorotris(triphenylphosphine)ruthenium(II) complex (RuCl₂(PPh₃)₃), and RuCl₃.

10. The process of claim 1, wherein the terminal alkene is a mixture of 2-propoxy-5-vinylcyclohexan-1-ol and 2-propoxy-4-vinylcyclohexan-1-ol, and the internal alkene is a mixture of 2-propoxy-5-ethylidenecyclohexan-1-ol and 2-propoxy-4- ethylidenecyclohexan-1-ol.

11. The process of claim 1, wherein the terminal alkene is methyl eugenol and the internal alkene is methyl isoeugenol.

12. The process of claim 1, wherein the terminal alkene is 9-decen-1-ol and the internal alkene comprises a mixture of 6-decen-1-ol, 7-decen-1-ol and 8-decen-1-ol.

13. The process of claim 1, wherein the isomerizing step is conducted in the absence of a solvent.

14. The process of claim 1 further comprising recovering the internal alkene.

15. The process of claim 1, wherein the temperature is in a range of from about 130° C. to about 240° C.
